(19) United States
(12) Patent Application Publication
(10) Pub. No.: US 2018/0184945 A1
(43) Pub. Date: Jul. 5, 2018

(54) METHOD AND DEVICE FOR DETECTING A WORSENING OF THE CARDIO- RESPIRATORY CONDITION OF A PATIENT WITHIN A RESPIRATORY ASSISTANCE DEVICE

(71) Applicants: Jean-Christian BOREL, Meylan (FR); Nellie TALEUX-EYSSAUTIER, St Martin D'Uriage (FR); Nathalie ARNOL, Saint Martin d'Hères (FR); Renaud TAMISIER, La Terrasse (FR); Jean Louis PÉPIN, Grenoble (FR)

(72) Inventors: Jean-Christian BOREL, Meylan (FR); Nellie TALEUX-EYSSAUTIER, St Martin D'Uriage (FR); Nathalie ARNOL, Saint Martin d'Hères (FR); Renaud TAMISIER, La Terrasse (FR); Jean Louis PÉPIN, Grenoble (FR)

(21) Appl. No.: 15/316,278

(22) PCT Filed: Jun. 4, 2015
(86) PCT No.: PCT/EP2015/062529

§ 371 Date: Dec. 5, 2016

(30) Foreign Application Priority Data
Jun. 5, 2014 (FR) 1401297

(51) Int. Cl. A61B 5/08; A61B 5/00; A61M 16/00

(52) U.S. Cl. CPC A61B 5/0816; A61B 5/746; A61B 5/0022; A61M 16/0051; A61M 16/0069; A61M 2205/3303; A61M 2205/3584; A61M 2205/3592; A61M 2230/42; A61M 2205/18; A61M 16/024

(57) ABSTRACT

A method and device for detecting a worsening of the cardiopulmonary state of a patient treated by a respiratory assistance device comprising means for gathering and processing parameters which are representative of
a first parameter that is representative of the patient's respiratory frequency over a first observation window, e.g. daily;
a second parameter that is representative of the percentage of cycles that were initiated by the patient during a second observation window, e.g. daily; and
a third parameter that is representative of the usage time of the device during a third observation window, e.g. daily.

The method is characterized in that it comprises the detection of a significant variation of one of said parameters for at least two consecutive or non-consecutive days within a consecutive period of n days, with n strictly greater than 3 and preferably ≥5; and in response thereto, generating an alert to the attention of said patient to inform him (or to the attention of a third party, such as a clinician) of a significant risk of worsening state cardiopulmonary.

### FIELD OF THE INVENTION

The present invention relates to the field of respiratory assistance devices for patients with respiratory insufficiency or suffering sleep apnea syndrome and particularly to a method and device for detecting a worsening of the respiratory condition of a patient treated by such a device.

### BACKGROUND OF THE INVENTION

Chronic respiratory failure and heart failure are interspersed with quick, successive aggravations (exacerbations) that lead to an accelerated worsening of the health condition of the patient. These iterative aggravations of the disease are associated with a poor prognosis and increased mortality. They are also the source of costly hospitalizations and impair the quality of life of the subjects. Thus the rate of re hospitalization for heart failure patients after a de-compensation is 40% at 30 days. These exacerbations are associated with symptomatic changes (Increased dyspnea and secretions . . . ) and physiological (changes in the characteristics of the ventilation of the patient, be it spontaneous or under the assistance of a respirator)

The non invasive ventilatory support under positive pressure on long-term home has become a treatment modality increasingly used in cardiac failure patients and chronic respiratory. The ventilatory support is based on the use of a medical device that is able to breathe air flow (with or without oxygen) in the airways of a patient to support partially or totally his respiratory effort. This device can be used either through a mask which is placed over the nose and/or mouth (noninvasive ventilatory support), or through a tracheotomy cannula (invasive ventilatory support).

This treatment with ventilatory support reduces these quick and brutal aggravations, improves the quality of life and, in the cases of some respiratory failures, the survival of the patient.

Recent conventional ventilatory support devices are fitted with software that provide details on daily use (patient compliance), and a number of ventilatory parameters measured during treatment (minute ventilation, tidal volume, respiratory rate, leaks, cycles initiated by the patient, etc.). The current telecommunications technologies allow these parameters to be uploaded to the clinician or home health service provider in charge of the patient.

However these conventional devices do not allow early detection of sudden worsening of cardio-respiratory condition of a patient, despite the large volume of data stored by them.

This is the problem addressed by the present invention.

### SUMMARY OF THE INVENTION

It is an object of the present invention to provide a method and device for early detection of the worsening of the cardiopulmonary state of a patient being treated by means of a ventilatory assistance device.

It is another object of the present invention to provide a method for easily upgrading known respiratory assistance devices by adding a function of early detection of the worsening of cardio-respiratory condition of a patient, particularly by taking advantage of possibilities of remote transmission and remote teleprocessing.

A third object of the present invention is to provide a process for performing a dynamic analysis of data from the respiratory assisting devices.

Those goals are achieved by means of a method of detecting a worsening of the cardiopulmonary state of a patient treated by a respiratory assistance device comprising means for gathering and processing parameters which are representative of
a first parameter that is representative of the patient's respiratory frequency over a first observation window, e.g. daily;
a second parameter that is representative of the percentage of cycles that were initiated by the patient during a second observation window, e.g. daily;
a third parameter that is representative of the usage time of the respiratory assistance device during a third observation window, e.g. daily;

The method is characterized in that it comprises the detection of a significant variation of one of said parameters for at least two consecutive or non-consecutive days within a consecutive period of n days, with n strictly greater than 3 and preferably ≥5; and

in response thereto, generating an alert to the attention of said patient to inform him (or to the attention of a third party, such as a clinician) of a significant risk of worsening state cardiopulmonary.

In one embodiment, the method is implemented directly within the respiratory assistance device, which is thus completely autonomous to generate alerts to the patient's attention.

Preferably, the method is implemented in a remote server receiving remote transmission parameters of the respiratory assistance device for the purpose of statistical analysis and ultimately generating a detailed warning upon detection a significant variation of one of the parameters for two consecutive days or non-consecutive during a consecutive period of n days, with n greater than three and preferably equal to 5 days.

In a particular embodiment, the warning signal takes the form of a light signal that is displayed on the respiratory assistance device or e-mail to an electronic mailbox or a SMS to a mobile phone, which will be the mobile phone of the patient, his doctor or any professional third party involved in the monitoring of the patient's health.

Preferably, the threshold values used for the detection of a potential increase of the parameters are quartiles which are continuously updated as new values and parameters are acquired by the process, and the alert is automatically generated upon detection of:
the occurrence during at least two consecutive or non-consecutive days over a period of at least n days, where n≥5, of an increase in the daily respiratory frequency beyond a value equal to the third quartile q3 calculated on the previous daily observation windows;
The occurrence during at least two consecutive or non-consecutive days over a period of at least n days, where n>5, of an increase in the percentage of daily cycles initiated by the patient beyond a value equal to the third quartile q3 of cycles percentages for patient-triggered calculated on the previous daily observation windows;
The occurrence during at least three consecutive or non-consecutive days over a period of at least n days, where n≥5, of one of the following two events:
   an increasing of the daily usage period beyond a value equal to the third quartile values q3 of the usage time calculated on the previous daily observation windows or
   a decrease of the duration of daily use below a value equal to the first quartile q1 of usage duration values calculated on the previous daily observation windows or a combination of the three above circumstances.
      or any combination of those three situations above.

The invention also allows the realization of a autonomous respiratory assistance device comprising:
a ventilator to support respiratory of a patient,
a control unit and treatment;
a measurement and sensing system for collecting data and parameters of the device, including:
a first parameter representative of the patient's respiratory frequency during a first observation window, e.g. daily;
a second parameter representative of the percentage of cycles triggered by the patient during a second observation window, e.g. daily;
a third parameter representative of the life of the device during a third observation window, e.g. daily;
a display unit (70)
detection means for detecting a significant variation of one of said parameters for at least two consecutive or non-consecutive days in a period of n consecutive days with n strictly greater than 3 and preferably ≥5; said detection means causing the generation of a warning to the attention of said patient to inform a significant risk of worsening his cardiopulmonary status.

Alternatively, the invention allows the achievement of a respiratory assistance device which is suitable for being integrated in a communication network, the device comprising communication means for, on one third hand, allowing the remote transmission of said parameters to a remote server and, on the other hand, the receiving of a control signal from said remote server for the purpose of generating an alert signal, sound or light, intended to alert the patient or practitioner responsible for the patient to a risk of worsening its cardiopulmonary status.

Finally, the invention allows the realization of a remote server adapted to communicate with a respiratory assistance device, the remote server being adapted to receive data from a remote server that are representative of the following parameters:
the patient's respiratory frequency during a first observation window, e.g. daily;
the percentage of cycles triggered by the patient during a second observation window, e.g. daily;
the usage time of the device during a third observation window, for example daily.

The server further comprises calculation means for detecting a significant change in one of said parameters for at least two consecutive or non-consecutive days within a consecutive period of n days, with n strictly greater than 3 and preferably ≥5; and in response thereto, generating an alert to the attention of said patient or to the attention of a practitioner to inform him of a significant risk of worsening of cardiopulmonary status of the patient.

### BRIEF DESCRIPTION OF THE DRAWINGS

Other features, objects and advantages of the invention will become apparent from reading the description and drawings below, given by way of non-limiting examples. In the accompanying drawings:

FIG. 1 shows a block diagram of one embodiment of a medical support device according to the present invention.

FIG. 2 illustrates a general architecture of a respiratory support medical device which is integrated in a network environment, allowing communication with a server via the Internet.

FIG. 3 illustrates one embodiment of the treatment method for the respiratory frequency parameter and the percentage of cycles initiated by the patient.

FIG. 4 illustrates an embodiment of a the treatment method for the compliance parameter or the duration of use of the respiratory assistance device.

FIG. 5 illustrates an example of data representative of the respiratory frequency and the detection of a so-called High value, being greater than the third quartile.

FIG. 6 illustrates an example of the computation of quartiles for the respiratory frequency being recorded by the embodiment described.

FIG. 7 illustrates an example of data being representative of the duration of use and the detection of HIGH and LOW values, being respectively higher than the third quartile Q3 and less than the first quartile Q1.

FIG. 8 shows an example of calculating the quartiles of the duration of use.

### DESCRIPTION OF A PREFERRED EMBODIMENT

It will now be described how one can improve the conventional respiratory or respiratory assistance devices so as to incorporate a valuable function which allows early detection of exacerbations for patients suffering chronic respiratory failure and sleep apnea syndrome.

In general, the invention can be adapted to any medical device for invasive ventilation or non invasive ventilation. As this is known by a skilled man, in the particular situation on Non Invasive Ventilation, NIV, one will not use the first endotracheal (intubation or tracheotomy).

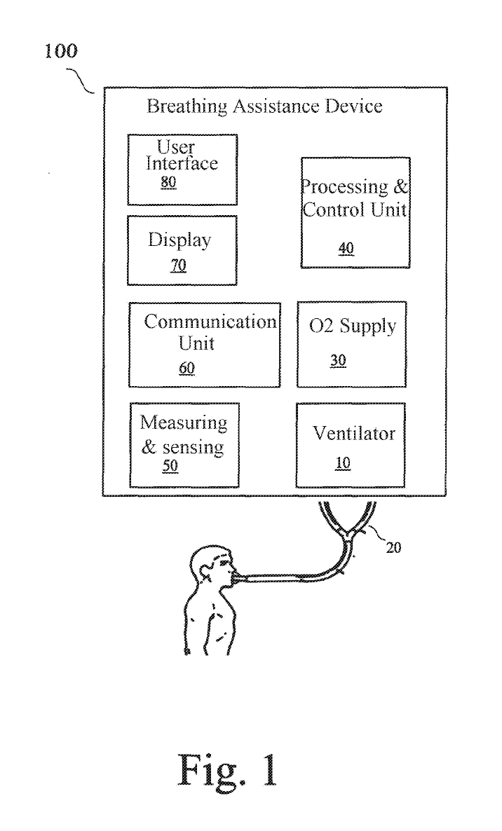

For purposes of illustration, such a respiratory assistance device is schematically shown in FIG. 1 under reference number 100, and includes a conventional ventilator 10, connected to a source of energy to operate autonomously or through a battery, optionally combined with a supply source of oxygen 30, under the control of a processing & control unit 40. The ventilator 10 blows air (enriched or not with oxygen) to the lungs of a patient through a conduit system 20 under the control of a microprocessor located within the processing & control unit 40, allowing different levels adjustments in the distribution of respiratory support and oxygen. In general, ventilator 10 is capable of operating in more conventional modes of operation well known to the art, it is not necessary to describe in more detail.

Device 100 further comprises a display 70 and optionally an user interface 80 for the patient, as well as a measuring & sensing system 50 which is provided with conventional means for measuring, in particular flow and/or pressure, for collecting extensive information and data to the control unit 40 and useful to the functioning of the respiratory support system 100 according to procedures well known to a skilled person.

In addition, measuring & sensing system 50 also allows, together with Processing & Control unit 40, the gathering of extensive information and ventilatory parameters which are measured (minute ventilation, tidal volume, respiratory frequency, leaks, cycles triggered by the patient, etc.) and that will be used in analytical processes for the early detection of an exacerbation. Those parameters include at least one of the three following parameters:
a first parameter representative of the respiratory frequency which is considered within a first observation window, e.g. daily;
   a second parameter representative of the percentage of cycles triggered by the patient within a second observation window, e.g. daily;
      a third parameter representative of the duration of use of the device during a third observation window, e.g. daily;

Conveniently, these parameters can be calculated in various ways, for instance based on an arithmetic mean or a median calculation.

As will be described below in detail, at least one of these three parameters can be advantageously used in an accurate statistical analysis for early detection of a potential exacerbation.

In a particular embodiment, the method of analysis and early detection described hereinafter is implemented locally, within the respiratory assistance device, and soliciting the data processing device that includes the processing & control unit 40 (processor, internal memory etc.).

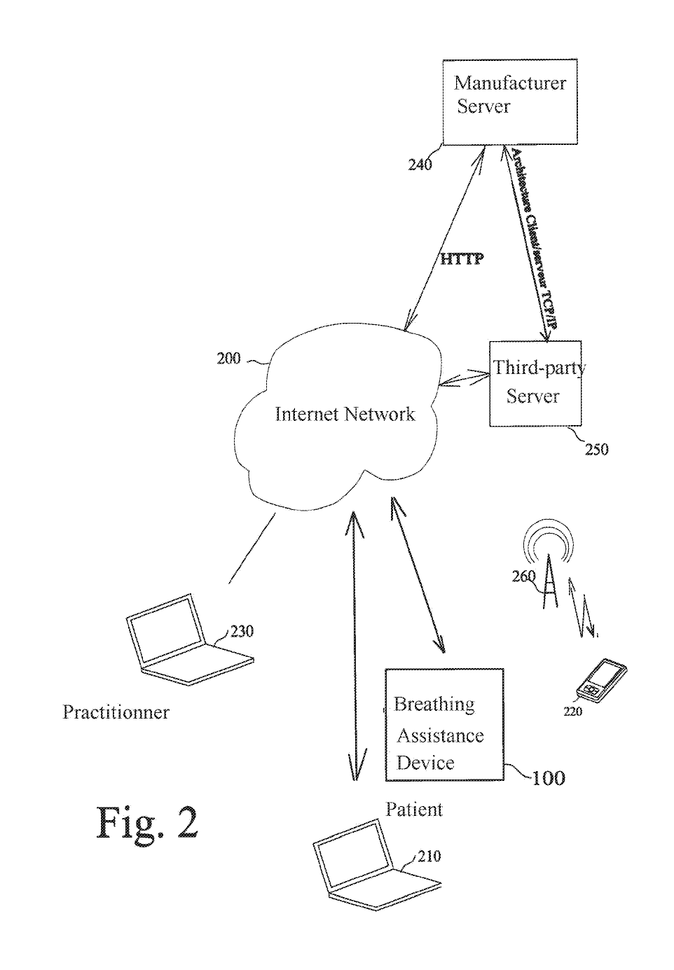

Preferably, however, the analysis and detection process includes a remote processing that is executed through a remote server—such as server 240 of FIG. 2 for example—based on the remote transmission of the above mentioned parameters thanks to communication unit 60 illustrated in FIG. 1. In its simplest form, this communication unit 60 may include a modem to access the Internet via the telephone line. In another embodiment, the communication unit may include a communication device in accordance with the IEEE 802.11 (Wi-Fi) allowing direct access to a wireless network and, consequently, to the Internet via a dedicated access point. Alternatively, the respiratory assistance device 100 may be provided with a communication unit based on the mobile phone, enabling a low-speed data communication (GSM, GPRS) or broadband (3G, 4G) as appropriate.

Generally speaking, communication unit 60 enables the transmission, in the uplink direction, of data and parameters used in the operation of the device and, in the downlink direction, the receiving of control instructions from a remote server.

FIG. 2 more particularly illustrates one embodiment of a respiratory assistance device 100 that is configured to be integrated within a wider telecommunications network, comprising the Internet network 200 with which device 100 is capable of communicating through communication unit 60, including access to a secure remote server 240, which may be the manufacturer of server device 100 or any remote server that may be used for storing and processing data collected within the device 100, possibly anonymously if the server has only the serial number of the device 100. More precisely, the communication between device 100 and server 240 may take various forms permitted by the various TCP/IP communication protocols and specifically, in case of need, the http protocol (shown in FIG. 2) or its secure version HTTPs which is widely used by Internet browsers.

In a particular embodiment, one may also consider the involvement of a third party server 250 communicating with the remote server 240 belonging to the manufacturer of the respiratory assistance medical device (eg which may communication via a Client/Server TCP/IP architecture) or may even communicate directly with the respiratory assistance medical device 100. The third party server 250 may be, for illustration but not limited to, the server of a medical-technical organization which serves as a support for an hospital and provides the latter with assistance and/or technical expertise for deploying respiratory assistance equipments to patients' homes.

Even more generally, the network can also integrate communication devices belonging to the patient, as illustrated in FIG. 2 showing a laptop 210 that directly communicates with the Internet and smartphone 220 which also allows a communication via the Internet thanks to the establishment of a data link with its base station 260.

In addition to the patent's equipment, the same network integrates communication tools of the practitioner or clinician, as shown by the computer 230 illustrated in FIG. 2.

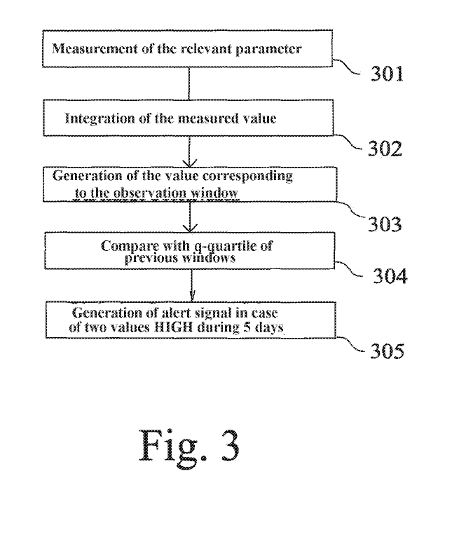

It will now be described, in relation to the FIGS. 3-8, how one can advantageously achieve a method for early detection of the exacerbation or worsening of the cardio-respiratory state of a patient while performing an accurate statistical analysis on one of the three following parameters collected by the measuring and sensing system 50 of the respiratory assistance device 100:
a first parameter representative of the respiratory frequency within a first observation window, e.g. daily;
a second parameter representative of the percentage of cycles triggered by the patient within a second observation window, e.g. daily;
a third parameter representative of the duration of use of the device within a third observation window, e.g. daily;

The process allows an early detection of the risk of exacerbation with the transmission of data to third parties responsible for the medical and paramedical monitoring of the patient, thus giving the patient an opportunity to quickly check the practitioner who will take the necessary preventive measures and reduce and, if necessary, the risk of a heavy and costly hospitalization.

To this end, the process leads to an automatic generation of a warning message to the attention of the patient or the practitioner or still any professional third party being authorized to receive such messages, upon detection of a significant variation of one of the three previous parameters for at least two consecutive or non-consecutive days within a consecutive period of n days, with n strictly greater than 3 and more particularly ≥5, preferably equal to five days.

In to determine a significant change in one of three parameters, the method proceeds as described below.

For the respiratory frequency and/or the percentage of cycles initiated by the patient, the method comprises the following steps, illustrated in FIG. 3:

Step 301: measuring the parameter considered, ie either the respiratory frequency or the percentage of cycles triggered;

Step 302: integration of the measured value of the parameter being considered in a population of values distributed in defined intervals q(q−1) q quantiles;

Step 303: generating a value representative of the parameter considered for the observation window considered, for example the daily window;

Step 304: comparison of the value generated in step 303 with the value of a q-quantile above the median for an earlier observation window, to determine potential HIGH value;

Step 305: generation of an alert signal or warning message in the case of a detection of two consecutive or non-consecutive values HIGH during a period of n consecutive days with strictly greater than 3 and preferably n≥5.

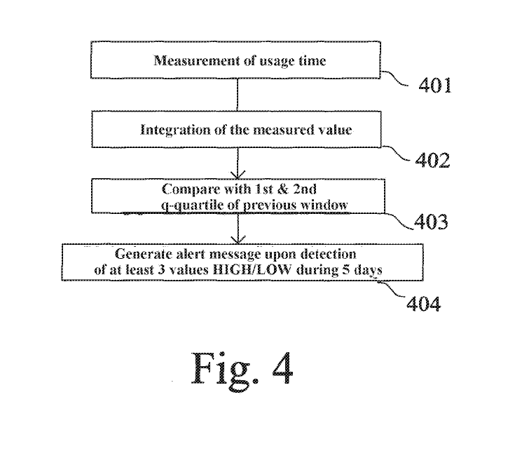

For the usage time, the method comprises the following steps, illustrated in FIG. 4:

Step 401: Measuring the usage time for the observation window considered, eg daily;

Step 402: integration of the value measured in a population of values which are distributed in q intervals defined by (q−1) q-quantiles;

Step 403: comparing the measured value for the observation window with the value of two quartiles corresponding to previous observation windows, respectively lower and higher than the median, for the determination of two values, respectively LOW and HIGH;

Step 404: generation of a alert signal or warning message upon detection of at least three values HIGH and/or LOW, consecutive or not, during a period of n days, with n>5.

Thanks to the process described above, the values measured and collected by measuring and sensing system 50 can be used to automatically generate an alert to the patient's care or its practitioner. The alert may take various forms, including the most varied in the context of a respiratory or respiratory assistance device 100 integrated in a communication network.

It may first be a simple visual or audible alert which is generated by device 100—e.g. visual broadcast message on display 70—in response to receiving a specific command issued by remote server 240 (or even third party server 250).

Alternatively, the system may generate an explicit email, sent on the email system 210 of the patient or that of his physician or received by SMS on the patient's or physician's mobile phone 220.

All adaptations will therefore be possible to increase patient responsiveness which will thus be invited to consult his/her practitioner as soon as possible.

Clearly, the three above described parameters can be advantageously combined to enhance the detection method and reduce false detections.

Preferably, the threshold values that are used for the detection of a potential increase of the parameters are percentiles of established reference values (75^{th} or higher than the 3^{rd} quartile) which are continuously updated while new values are continuously collected by the system, and the alert or warning message is automatically generated upon detection of an increase/decrease beyond the threshold values.

In a preferred embodiment which will now be more particularly described, the observation window is a daily window and the population of values that is measured is distributed into quartiles, so that the alert is automatically generated upon detection of the one of the three following situations:
an occurrence during at least two consecutive or non-consecutive days within a period of n days, preferably with n≥5, of an increase of daily respiratory frequency beyond a value equal to the third quartile Q3 calculated on previous daily observation windows;
an occurrence during at least two consecutive or non-consecutive days within a period of n days, preferably with n≥5, an increase in the percentage of daily cycles triggered by the patient beyond a value equal to the third quartile q3 of the values of cycles percentages triggered by the patient and calculated on the previous daily observation windows;
the occurrence during at least three consecutive or non-consecutive days over a period of n days, with n≥5, of one of the following two events:
   an increase of the daily usage duration beyond a value equal to the third quartile q3 of the usage duration values calculated on previous daily observation windows or
   a decrease of the daily usage time below a value equal to the first quartile q1 of the usage duration values calculated on the previous daily observation windows

Any combination of these three circumstances may usefully be considered to make more robust the process of early detection and reduce, if any, false detections.

In the preferred embodiment, a server 240 (or 250) is also configured and specially arranged for communicating with a respiratory assistance device as described above and illustrated in FIG. 1, and adapted to receive through electronic remote transmission the following parameters that are representative:
The patient's respiratory frequency during a first observation window, eg daily;
The percentage of cycles triggered by the patient for a second observation window, eg daily;
The usage duration of the device during a third observation window, for example daily.

The server 240 (or 250) comprises a Computing unit for processing the collected values above, so as to detect a significant variation of one of the three parameters for two days (or three) consecutive or non-consecutive during a consecutive period n days, n=5 days, for example.

Upon this detection, the server is configured to generate an alert message to the respiratory support device 100, or email/SMS to one of systems 210-220-230.

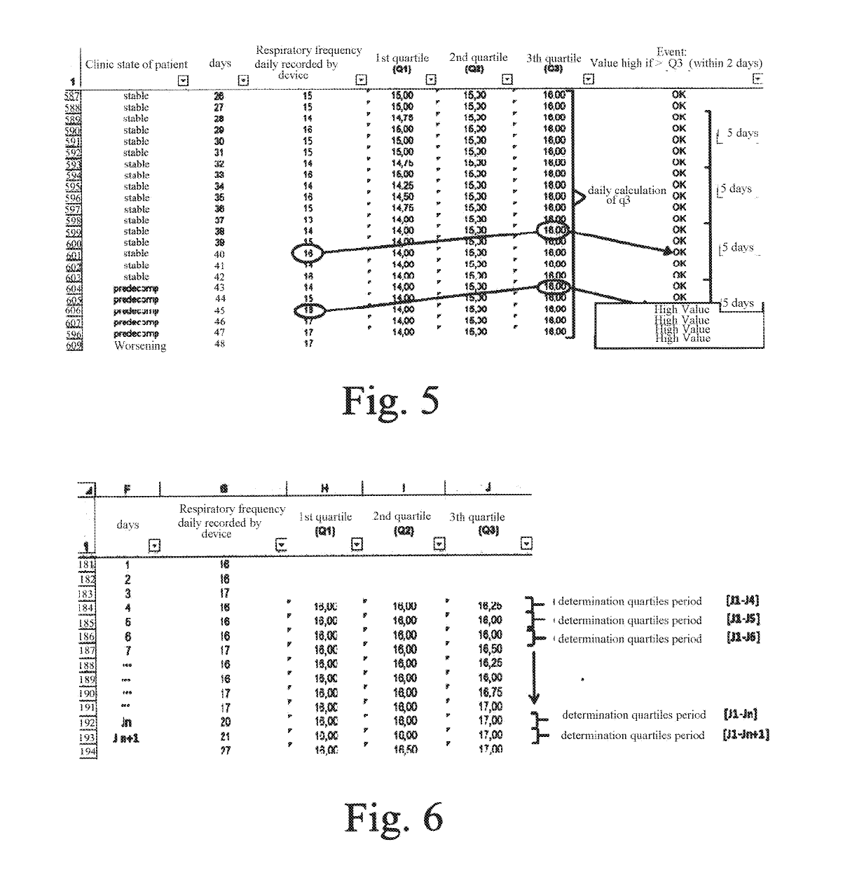

FIG. 5 illustrates an example of data representative that are representative of the respiratory frequency and which further illustrates the detection of a so-called HIGH value, which is greater than the third quartile. The respiratory frequency which is recorded by such devices corresponds to the number of times/minute where the device generates a flow during insufflation to the patient. This respiratory frequency, expressed in cycles/minute, is expressed by the average or median value per day based on different manufacturers on the market. The method includes detecting a respiratory frequency that will be classified as “HIGH” value at least two days (consecutive or not) during a period of five days. A respiratory frequency is classified to be a “HIGH” value one day if it is greater than the third quartile of respiratory frequencies recorded by the device until the day before, as shown in FIG. 5.

FIG. 6 illustrates an example of the computation of quartiles of the respiratory frequency recorded by the device in the embodiment described. One sees that the calculation of the 3^{rd} quartile of the respiratory frequency requires at least four consecutive days of record. It is then recalculated daily, taking into account each day the device is being used. This is, however, only one non limiting embodiment.

In a similar manner, one proceeds to analyze the percentage of cycles initiated by the patient. As described above, the respiratory frequency that is recorded by the ventilatory assistance devices corresponds to the number of cycles/mn that the device produces for the patient. Each cycle can either be triggered by the patient himself, or initiated by the device if the patient's triggering rate is less than the frequency being programmed in the unit.

The ratio of the number of cycles triggered by the patient on the number of total cycles executed by the device represents “the percentage of triggered cycles.” This variable is reported (mean or median) per day of use. For example, one patient that triggers all the respiratory cycles of the device will have a percentage of triggered cycles being equal to 100%. A patient which triggers half of the cycles while the other half is automatically generated by the device will have a percentage cycles triggered to 50%.

The percentage of cycles triggered by the patient is analyzed as for the respiratory frequency described above the method includes detecting the percentage of triggered cycles that will be classified as a “HIGH” value at least two days (consecutive or not) by five days. The percentage of triggered cycles is ranked to be a so-called “HIGH” value one day if it is higher than the third quartile of the cycles triggered percentages recorded by the device until the day before. Computing the third quartile of the percentage of cycles triggered is the same as the respiratory frequency.

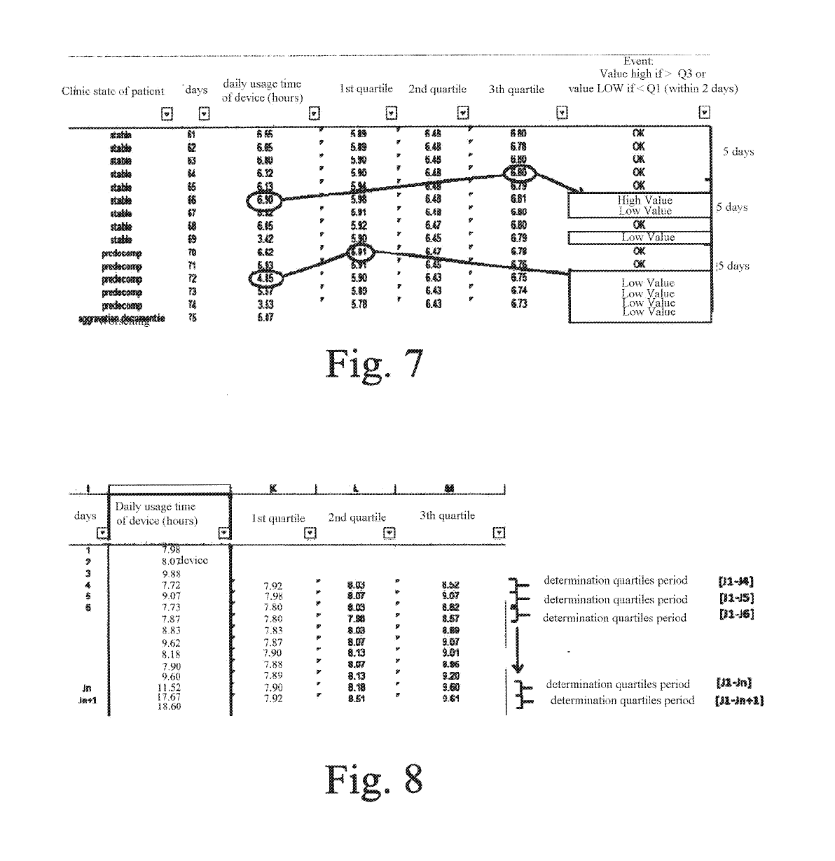

FIG. 7 illustrates an example of data representative of the usage time and the detection of so-called HIGH and LOW values, respectively greater than the third quartile Q3 and less than the first quartile Q1. Practically, the daily usage time is recorded by the ventilatory support devices calendar). It is reported in hours: minutes/day.

The daily usage time is analyzed as previously described and, as seen in FIG. 7, comprises detecting a period of daily use being “HIGH” or “LOW” at least two days—and preferably three days—consecutive or not—over a duration of five days at least.

In the embodiment shown in FIG. 7, the daily duration of use is assigned the value “HIGH” one day if it is greater than the third quartile of daily usage times recorded by the device until the day before. The daily duration of use is assigned a value “LOW” one day when it is lower than the first quartile of the daily duration of use recorded by the device until the day before (FIG. 7).

The computation of the 1^{st} and 3^{rd} quartiles of the duration of use is identical to that of the respiratory frequency discussed above, and is more specifically illustrated in FIG. 8.

The three parameters described above—respiratory frequency, percentage of triggered cycles and duration of use, are by no means exclusive to the possibility of combination with other parameters that can be defined and identified to achieve the same goal.

More generally, the skilled man can extend the method and apparatus described and slightly amend the values of quartiles q1 and q3, ie corresponding to the two percentile thresholds respectively between [20, 30] and [70, 80].

What is claimed is:

1. A method Of detecting a worsening of the cardiopulmonary state of a patient treated by a respiratory assistance device comprising a ventilator connected to a power source, associated with oxygen supply source under the control of a control and processing unit, said ventilator blowing air enriched or not with oxygen to the lungs of a patient through a conduit system that is under control of a microprocessor located within said control unit,
said respiratory assistance device comprising means for collecting and processing parameters and data representative of:
a first parameter that is representative of the patient's respiratory frequency over a first observation window;
a second parameter that is representative of the percentage of cycles that were initiated by the patient during a second observation window; and
third parameter that is representative of the duration of use of the device during a third observation window;
said method comprising the steps of detecting a significant variation of one of said parameters for at least two consecutive or non-consecutive days within a consecutive period of n days, with n strictly greater than 3 and preferably ≥5; and
in response thereto, generating in said control and processing device a warning for the attention of said patient or practitioner to inform a significant risk oaf aggravation of his condition cardiopulmonary.

2. The method according to claim 1, wherein said first, second and third observation windows are daily.

3. The method according to claim 1, wherein said method is implemented within said respiratory assistance device.

4. The method according to claim 1, wherein said method is implemented in a remote server receiving daily said parameters of said respiratory assistance device for statistical analysis.

5. The method of claim 4, wherein said alert takes the form of a warning signal, for example a light which is displayed on said respiratory assistance device, or an email, or a text message transmitted to an email address or a predetermined number.

6. The method according to claim 1, wherein said alert is automatically generated upon detection of at least one of:
the occurrence during at least two consecutive or non-consecutive days over a period of at least n days, where n≥5, of an increase in the daily respiratory frequency beyond a value equal to the third quartile q3 calculated over the previous daily observation windows;
the occurrence during at least two consecutive or non-consecutive days over a period of at least n days, where n≥5, of an increase in the percentage of daily cycles initiated by the patient beyond a value equal to the third quartile q3 cycles percentages for patient-triggered cycles calculated on the previous daily observation windows; and
the occurrence during at least three consecutive or non-consecutive days over a period of at least n days, where n≥5, of one of the following two events:
an increase of the daily use time beyond a value equal to the third quartile q3 of usage time values calculated on previous daily observation windows or
a decrease of the daily use time below a value equal to the first quartile q1 of the use time values calculated on the previous daily observation windows or any combination of the three above circumstances.

7. A respiratory assistance device for carrying out the method recited in claim 1, said device comprising, in combination:
a ventilator for supporting the respiration of a patient,
a control and processing unit;
a measurement and sensing system for the collection of data and parameters of the device, including:
a first parameter representative of the respiratory frequency on a first observation window;
a second parameter representative of the percentage of cycles initiated by the patient during a second observation window;
a third parameter representative of the use time of the apparatus during a third observation window;
a display unit; and
means for detecting a significant variation of one of said parameters during two consecutive or non-consecutive days over a period of n consecutive days with n strictly greater than 3, preferably ≥5; said detecting means causing the generation of a warning to the attention of said patient to inform a significant risk of Worsening said patient's cardiopulmonary status.

8. The device according to claim 7, wherein said measurement and sensing system is arranged so that said first, second and third observation windows of said parameters collected by said measurement and sensing system are daily.

9. The device according to claim 7, wherein said control and processing unit is arranged so that said warning is generated automatically by said control and processing unit upon detection of:
the occurrence during at least two consecutive or non-consecutive days over a period of at least n days, where n≥5, of an increase in the daily respiratory frequency beyond a value equal to the third quartile q3 calculated on the previous daily observation windows,
the occurrence during at least two consecutive or non-consecutive days over a period of at least n days, where n≥5, of an increase in the percentage of daily cycles initiated by the patient beyond a value equal to the third quartile q3 of percentage of daily cycles triggered calculated on the previous daily observation windows; and
the occurrence during at least three consecutive or non-consecutive days over a period of at least n days, where n≥5, of one of the following two events:
an increase of the daily usage time beyond a value equal to the third quartile q3 of values of usage time calculated on previous daily observation windows, or
a decrease in the daily usage time below a value equal to the first quartile q1 of usage times calculated on the previous daily observation windows or a combination of the three above circumstances.

10. The respiratory assistance device according to claim 9, the first quartile q1 is replaced by a first threshold in the range of percentiles and wherein the third quartile q3 is replaced by a second threshold included in percentiles of the range.

11. A respiratory assistance device for carrying out the method recited in claim 4, comprising, in combination:
a ventilator for supporting the respiration of a patient;
a control and processing unit;
a measurement and sensing system for the collection parameters, and operative data representative of the working of the device, including:
a first parameter representative of the patient's respiratory frequency during a firs observation window;
a second parameter representative of the percentage of cycles initiated by the patient during a second observation window;
a third parameter representative of the usage duration of the apparatus during a third observation window;
a display unit, and
communication means serving firstly the remote transmission of said parameters to a remote server and, on the other hand, receiving a control signal from said remote server for generating a signal alert, sound or light, intended to alert the patient or practitioner of a risk of worsening of his cardiopulmonary status.

12. Device according to claim 11, wherein said first, second and third observation windows of said parameters collected by said sensing system are daily windows.

13. A remote server suitable for communication with a respiratory assistance device, the remote server being adapted to the reception by remote transmission representative of one or more of the following parameters:
the patient's respiratory frequency during a first observation window;
the percentage of cycles initiated by the patient during a second observation window; and
the duration of use of the device during a third observation window;
said server comprising calculation means for determining the detection of a significant variation of one of said parameters for at least two consecutive or non-consecutive days within a consecutive period of n days, with n greater than 3 and preferably ≥5; and
in response thereto, generating an alert to the attention of said patient to inform a significant risk of worsening his cardiopulmonary status.

14. The remote server according to claim 13, wherein the alert takes the form of an email, a text message or an electronic message sent to the patient's respiratory assistance device.

15. The remote server according to claim 13, wherein the alarm takes the form of an email, an SMS message or an electronic message sent on a data processing device, such as a computer or a telephone mobile, the patient or his physician.
